(19) United States
(12) Patent Application Publication
(10) Pub. No.: US 2003/0027874 A1
(43) Pub. Date: Feb. 6, 2003

(54) Metal oxide-containing catalysts and use thereof in fischer-tropsch processes

(75) Inventors: Norman Herron, Newark, DE (US); Kostantinos Kourtakis, Media, PA (US); Munirpallam A. Subramanian, Kennett Square, PA (US); Olga Ionkina, Ponca City, OK (US); Nithya Srinivasan, Ponca City, OK (US)

Correspondence Address: DAVID W. WESTPHAL, CONOCO PHILLIPS, P.O. BOX 4783, HOUSTON, TX, 77210-4783, US

(73) Assignee: Conoco Inc., Houston, TX

(21) Appl. No.: 10/184,472

(22) Filed: Jun. 27, 2002

Related U.S. Application Data
(60) Provisional application No. 60/301,711, filed on Jun. 28, 2001.

(51) Int. Cl. C07C 27/06

(52) U.S. Cl. 518/713; 518/714; 518/715

(57) ABSTRACT

A catalyst and process for producing hydrocarbons using the catalyst is provided. The process involves contacting a feed stream comprising hydrogen and carbon monoxide with the catalyst in a reaction zone maintained at conversion-promoting conditions effective to produce an effluent stream comprising hydrocarbons. In accordance with this invention the catalyst used in the process includes at least one catalytic metal for Fischer-Tropsch reactions, preferably cobalt. The catalyst further includes a structural component, preferably a support, that includes a metal selected from the group consisting of oxides of Group 2 metals, Group 3 metals, Group 6 metals, Group 8 metals, Group 12 metals, Group 15 metals, and combinations thereof, preferably as the oxide.

### RELATED APPLICATIONS

The present application claims the benefit of 35 U.S.C. 111(b) provisional application Serial. No. 60/301,711 filed Jun. 28, 2001, and entitled Metal-Oxide-Containing Catalysts and Use Thereof in Fischer-Tropsch Processes.

### STATEMENT REGARDING FEDERALLY SPONSORED RESEARCH OR DEVELOPMENT

Not Applicable.

### TECHNICAL FIELD OF THE INVENTION

The present invention relates generally to the field of Fischer-Tropsch reactions for the catalytic production of hydrocarbons from synthesis gas, a mixture of carbon monoxide and hydrogen. More particularly, the present invention relates to metal oxide supports, metal oxide supported catalysts, preferably cobalt-based catalysts, and the use of the catalysts for the production of variety of hydrocarbons from CH₄ to higher hydrocarbons and aliphatic alcohols, preferably long chain length hydrocarbons in the diesel weight range. Still more particularly, the metal is preferably at least one of a Group 2 metal, a Group 3 metal, a Group 6 metal, Fe, a Group 12 metal, a Group 15 metal, and combinations thereof, more preferably at least one of Ca, La Ce, Cr, Fe, Zn, and Bi, and combinations thereof.

### BACKGROUND OF THE INVENTION

Liquid hydrocarbons serve a number of important purposes and are an invaluable source of gasoline and diesel fuel. Historically, such hydrocarbons have been obtained through drilling and extraction from oil reserves. Unfortunately, though, these reserves represent an exhaustible supply that is quickly being depleted. Alternatively, liquid hydrocarbons can be synthesized from natural gas, a mixture of short-chain hydrocarbons including principally methane. As the oil reserves are depleted, this approach is becoming an increasingly attractive method of acquiring longer chain hydrocarbons, in part because the natural gas reserve is expected to significantly outlast the remaining oil reserves.

The conversion of methane to hydrocarbons is typically carried out in two steps. In the first step, methane is converted into a mixture of carbon monoxide and hydrogen, commonly referred to as synthesis gas or syngas. In a second step, the synthesis gas is converted into various hydrocarbons. This second step, the preparation of hydrocarbons from synthesis gas, is well known in the art and is usually referred to as a Fischer-Tropsch synthesis, Fischer-Tropsch process, or Fischer-Tropsch reaction. Fischer-Tropsch synthesis generally entails contacting a stream of synthesis gas with an appropriate catalyst under temperature and pressure conditions that favor the formation of hydrocarbon products. The product stream prepared by using these catalysts usually includes a mixture of hydrocarbons having a very wide range of molecular weights. Product distribution or product selectivity depends heavily on the type and structure of the catalysts and on the reactor type and operating conditions. Accordingly, it is highly desirable to maximize the productivity and selectivity of the Fischer-Tropsch synthesis to the production of high-value liquid hydrocarbons.

Catalysts for use in the Fischer-Tropsch synthesis usually contain a catalytic metal of Groups 8, 9, or 10 (in the new notation of the periodic table of the elements, which is followed throughout). In particular, iron, cobalt, nickel, and ruthenium have commonly been used as the catalytically active metals. Nickel catalysts favor termination and are useful for the selective production of methane from synas. Iron has the advantage of being readily available and relatively inexpensive but the disadvantage of a relatively low catalyst activity. Ruthenium has the advantage of high activity but unfortunately is quite expensive. Consequently, although ruthenium is not the economically preferred catalyst for commercial Fischer-Tropsch production, it is often used in low concentrations as a promoter with one of the other catalytic metals. Cobalt has the advantages of being more active than iron and more economically feasible than ruthenium. Further, cobalt is less selective to methane than nickel.

Accordingly, cobalt has been extensively investigated as a catalyst for the production of hydrocarbons with weights corresponding to the range of the gasoline, diesel, and higher weight fractions of crude oil. In particular, cobalt has been found to be suitable for catalyzing a process in which synthesis gas is converted to hydrocarbons having primarily five or more carbon atoms (i.e., where the C₅₊ selectivity of the catalyst is high). Depending on the molecular weight product distribution, different Fischer-Tropsch product mixtures are ideally suited to different uses. For example, Fischer-Tropsch product mixtures containing C₅₊ hydrocarbons may be processed to yield gasoline, as well as heavier middle distillates. Further, Fischer-Tropsch product mixtures containing primarily C₁₁₊ hydrocarbons are also useful for further processing to yield middle distillates. Middle distillates typically include heating oil, diesel fuel, and kerosene. C₂₀₊ hydrocarbons are typically hydroprocesses to yield a lighter product, such as gasoline or middle distillates. See, for example, H. Schulz, Short History and Present Trends of Fischer-Tropsch Synthesis, APPLIED CATALYSIS A, vol.186, pp.3-12 (1999), which is hereby incorporated herein by reference in its entirety.

Catalyst systems often employ a promoter in conjunction with the principal catalytic metal. A promoter typically improves a measure of the activity of a catalyst, such as productivity, selectivity, lifetime, reducibility, or regenerability. Ruthenium, rhenium, and combinations thereof, are widely known as promoters for cobalt-based Fischer-Tropsch catalysts. However, ruthenium and rhenium are each rare and costly. Thus, although these promoters are used at relatively low concentrations, they contribute significantly to the cost of Fischer-Tropsch catalysis.

Catalysts conventionally include a support material. The support material serves as a carrier for the catalytic metal and any promoters deposited on the support and is typically porous. Catalyst supports for catalysts used in Fischer-Tropsch synthesis of hydrocarbons have typically been refractory oxides (e.g., silica, alumina, titania, thoria, zirconia or mixtures thereof, such as silica-alumina).

With respect to supported cobalt-based catalysts, reference is made to the following patents. U.S. Pat. No. 4,542,122 discloses a cobalt or cobalt-thoria on titania having a preferred ratio of rutile to anatase, as a hydrocarbon synthesis catalyst. U.S. Pat. No. 4,088,671 discloses a cobalt-ruthenium catalyst where the support can be an inorganic metal oxide, preferably alumina for economic reasons. U.S. Pat. No. 4,413,064 discloses an alumina supported catalyst having cobalt, ruthenium and a Group 3 or Group 4 metal oxide, e.g., thoria. European Patent 142,887 discloses a silica supported cobalt catalyst together with zirconium, titanium, ruthenium and/or chromium.

Research continues on the development of more efficient but lower cost Fischer-Tropsch catalyst systems and reaction systems that increase the selectivity for high-value hydrocarbons in the Fischer-Tropsch product stream. Despite the vast amount of research effort in this field, there is still a great need for new economical catalyst systems for Fischer-Tropsch synthesis that will provide improved selectivity toward longer-chain hydrocarbons. In particular, Fischer-Tropsch systems are needed that have improved yields of hydrocarbons having eleven or more carbon atoms without the need for expensive catalyst metals or promoter materials.

### SUMMARY OF THE INVENTION

According to an embodiment, the present invention features a catalyst that preferably has a nominal composition of MₐO_{b}/N_{c}O_{d}, where a is preferably between 1 and 6, more preferably between 1 and 3, b is preferably between 1 and 6, more preferably between 1 and 4, c is preferably between 1 and 3, more preferably between 1 and 2, and d is preferably between 1 and 4, more preferably between 1 and 3. N includes a first metal selected from the group consisting of Group 2 metals, the Group 3 metals, the Group 6 metals, the Group 8 metals, the Group 12 metals, the Group 15 metals, and combinations thereof, preferably from the group consisting of zinc and chromium, and combinations thereof. M includes a second metal, preferably a catalytic metal, more preferably, a Fischer-Tropsch catalytic metal, more preferably an iron-group metal, most preferably cobalt. M may further include an optional promoter.

According to another embodiment, the present invention features a catalyst that preferably includes a support that includes an oxide of a metal selected from the group consisting of Group 2 metals, Group 3 metals, Group 8 metals, Group 12 metals, Group 15 metals, and combinations thereof. The metal oxide is preferably selected from among zinc oxide and chromia. The catalyst preferably further includes a catalytic metal, preferably deposited on the support. When the catalyst includes a catalytic metal, the catalyst preferably is made by impregnating the support with the catalytic metal. The catalyst may further include an optional promoter.

According to some embodiments, a Fischer-Tropsch process includes contacting a feed stream including hydrogen and carbon monoxide with a catalyst according to any one of the above-described embodiments in a reaction zone that is maintained at conversion-promoting conditions effective to produce an effluent stream that includes hydrocarbons.

In any one of the above-described embodiments, the hydrocarbons may have a weight range suitable for processing to diesel fuel. In particular, the hydrocarbons may include hydrocarbons having eleven or more carbon atoms that are suitable for processing to diesel fuel. Alternately, the hydrocarbons may have a weight range suitable for processing to gasoline. In particular, the hydrocarbons may include hydrocarbons having five or more carbon atoms.

In some embodiments, a catalyst according to the above-described embodiments includes an un-promoted catalytic metal and the catalyst has an activity of at least the activity of a comparative catalyst promoted with rhenium, ruthenium, or combinations thereof.

The present invention comprises a combination of features and advantages that enable it to overcome various selectivity problems of prior catalysts and processes. The various characteristics described above, as well as other features, will be-readily apparent to those skilled in the art upon reading the following detailed description of the preferred embodiments of the invention. It will be understood that as contemplated herein, a yield, e.g. the C₅⁺ productivity or the C₁₁⁺ productivity, of a catalyst containing a promoter according to the preferred embodiments of the present invention may be measured in any conventional units, e.g. gram·product per hour per liter (reactor volume) or gram·product per hour per kg·catalyst.

### DETAILED DESCRIPTION OF THE PREFERRED EMBODIMENT

According to a preferred embodiment of the present invention, a catalyst includes at least one metal, denoted N herein, selected from among the Group 2 metals, the Group 3 metals, the Group 6 metals, the Group 8 metals, the Group 12 metals, the Group 15 metals, and combinations thereof. The catalyst includes more preferably at least one metal selected from among Ca, La, Ce, Cr, Fe, Zn, and Bi, and combinations thereof, most preferably at least one metal selected from among zinc and chronium, and combinations thereof. The metal is preferably present in the form of a compound, preferably an oxide. The metal may be a mixture of more than one of the above-described metals. Thus, the oxide may be a mixed metal oxide. The catalyst preferably further includes another metal, denoted M herein, preferably a metal selected from among Group 8 metals, Group 9 metals, and Group 10 metals, and combinations thereof, preferably in an amount catalytically active for the Fischer-Tropsch synthesis. M is preferably selected from among the iron-group metals, most preferably cobalt. M may further include any optional promoters. Thus, the catalyst preferably has a nominal composition of MₐO_{b}/N_{c}O_{d}, where a is preferably between 1 and 6, more preferably between 1 and 3, b is preferably between 1 and 6, more preferably between 1 and 4, c is preferably between 1 and 3, more preferably between 1 and 2, and d is preferably between 1 and 4, more preferably between 1 and 3.

The metal N, or compound containing N, such as an oxide of N, preferably acts as a structural material. That is, the metal is preferably present in a structural component of the catalyst. In particular, the catalyst preferably has a composition that includes at least 50% by weight of a structural component, where the structural component includes at least one of the above-described metals, more preferably an oxide of at least one of the above-described metals. The structural component is preferably in the form of a catalyst support. The support is preferably a porous carrier material, more preferably having a surface suitable for receiving deposited catalytic metal.

According to an embodiment of the present invention, a catalyst includes a support that includes an oxide of a Group 2 metal or a combination of Group 2 metals. The Group 2 metals include Be, Mg, Ca, Sr, and Ba. Thus, the support may include a beryllium oxide, such as BeO (beryllium monoxide, occurring naturally in bromellite), and the like. Alternatively, the support may include a magnesium oxide, such as MgO (magnesium monoxide, occurring naturally in periclase), MgO₂ (magnesium peroxide), and the like. Still alternatively, the support may include a calcium oxide, such as CaO (calcium monoxide) and CaO₂ (calcium dioxide), and the like. Yet alternatively, the support may include a strontium oxide, such as SrO (strontium monoxide), SrO₂ (strontium peroxide), and the like. Still yet alternatively the support may include a barium oxide, such as BaO (barium monoxide), BaO₂ (barium peroxide), and the like.

According to another embodiment of the present invention, a catalyst includes a support that includes an oxide of a Group 3 metal or a combination of Group 3 metals. The Group 3 metals include Sc, Y, the Lanthanides, and the Actinides The Lanthanides include elements with atomic numbers 57-71 inclusive. The Actinides include elements with atomic numbers 89 and above inclusive. Thus, the support may include a scandium oxide, such as Sc₂O₃ (scandium sesquioxide, also termed scandia), and the like. Alternatively, the support may include an yttrium oxide, such as Y₂O₃ (yttrium sesquioxide, also termed yttria), and the like. Still alternatively, the support may include a lanthanum oxide, such as La₂O₃, (lanthanum sesquioxide, also termed lanthana), and the like. Yet alternatively, the support may include a cerium oxide, such as CeO₂ (cerium(IV) dioxide, also termed ceria), Ce₂O₃ (cerium(III) sesquioxide), and the like. Still yet alternatively, the support may include another oxide of a lanthanide, such as PrO₂ (praseodymium dioxide), Pr₂O₃ (praseodymium sesquioxide, also termed praseodymia), and the like. Yet still alternatively, the support may include an oxide of an actinide, such as ThO₂ (thorium dioxide, also termed thorianite), and the like.

According to still another embodiment of the present invention, a catalyst includes a support that includes an oxide of a Group 6 metal or a combination of Group 6 metals. The Group 6 metals include Cr, Mo, and W. Thus, the support may include a chromium oxide, such as CrO₂ (chromium dioxide), CrO₃ (chromium trioxide), CrO (chromium(II) monoxide), Cr₂O₃ (chromium(III) sesquioxide), and the like. Alternatively, the support may include a molybdenum oxide, such as MoO₂ (molybdenum dioxide), Mo₂O₅ (molybdenum pentoxide), Mo₂O₃ (molybdenum sesquioxide), MoO₃ (molybdenum trioxide), and the like. Still alternatively, the support may include a tungsten oxide, such as WO₂ (tungsten dioxide), W₂O₅ (tungsten pentoxide, also termed mineral blue), WO₃ (tungsten trioxide), and the like.

According to yet another embodiment of the present invention, the catalyst includes a Group 8 metal or a combination of Group 8 metals. The Group 8 metals include Fe, Ru, and Os. Thus, the support may include an iron oxide, such as FeO (iron(II) oxide, also termed ferrous oxide, and occurring naturally in wuestite), Fe₂O₃ (iron(III) oxide, also termed ferric oxide, and occurring naturally in hematite and magnetite), and the like. Alternatively, the support may include a ruthenium oxide, such as RuO₂ (ruthenium dioxide), Ru₂O₄ (ruthenium tetroxide), and the like. Still alternatively, the support may include an iridium oxide, such as IrO₂ (iridium dioxide), Ir₂O₃ (iridium sesquioxide), and the like.

According to still yet another embodiment of the present invention, the catalyst includes a Group 12 metal or a combination of Group 12 metals. The Group 12 metals include Zn, Cd, and Hg. Thus, the support may include a zinc oxide, such as ZnO (zinc monoxide), and the like. Alternatively, the support may include a cadmium oxide, such as CdO (cadmium monoxide) and the like. Still alternatively, the support may include a mercury oxide, such as Hg₂O (mercury(I) oxide), HgO (mercury(II) oxide, occurring naturally in montroydite), and the like.

According to yet still another embodiment of the present invention, the catalyst includes a Group 15 metal. The Group 15 metals include As, Sb, and Bi. Thus, the support may include an arsenic oxide, such as As₂O₅ (arsenic pentoxide), As₂O₃ (arsenic trioxide, occurring naturally in arsenolite and claudetite), and the like. Alternatively, the support may include an antimony oxide, such as Sb₂OS (antimony pentoxide), Sb₂O₄ (antimony tetroxide, occurring naturally in cervantite), Sb₂O₃ (antimony trioxide, occurring naturally in senarmonite and valentinite), and the like. Still alternatively, the support may include a bismuth oxide, such as BiO (bismuth monoxide), Bi₂O₃ (bismuth trioxide), BiO₅ (bismuth pentoxide), and the like.

A catalyst according to a preferred embodiment of the present invention preferably further includes a catalytic metal. The catalytic metal is preferably selected from the iron-group metals (i.e. cobalt, iron, and nickel), and combinations thereof. The catalytic metal preferably includes cobalt, and more preferably is essentially cobalt. The catalyst preferably contains a catalytically effective amount of the catalytic metal. The amount of catalytic metal present in the catalyst may vary widely. When the catalytic metal is cobalt, the catalyst preferably includes cobalt in an amount totaling from about 1% to 50% by weight (as the metal) of total catalyst composition (catalytic metal, support, and any optional promoters), more preferably from about 5% to 40% by weight, still more preferably from about 10 to about 37 wt. % cobalt, sill yet more preferably from about 15 to about 35 wt. % cobalt. It will be understood that % indicates percent throughout the present specification.

The catalytic metal contained by a catalyst according to a preferred embodiment of the present invention is preferably in a reduced, metallic state before use of the catalyst in the Fischer-Tropsch synthesis. However, it will be understood that the catalytic metal may be present in the form of a metal compound, such as a metal oxide, a metal nitrate, a metal hydroxide, and the like. The catalytic metal is preferably dispersed on the support. Although the catalytic metal may diffuse into the support, it is preferable that the catalytic metal is primarily present at the surface of the support, in particular on the surface or within a surface region of the support. That is, the catalyst preferably includes a surface region and an interior region, where the interior region contains primarily the bulk of the support and the surface region includes the surface of the support and may contain materials, such as a catalytic metal, at the surface of the support. It will be understood that a surface material may diffuse into the bulk of the support. However, the interior region and the surface region may be readily identified by conventional spectroscopic methods, such as infra red (IR) spectroscopy, nuclear magnetic resonance (NMR) spectroscopy, Auger electron spectroscopy (AES), x-ray photoelectron spectroscopy (XPS), extended x-ray absorption fine structure (EXAFS) spectroscopy, secondary ion mass spectrometry (SIMS), and the like. In particular, the presence of catalytic metal at the surface of the catalyst may be observed by XPS and EXAFS, and the like.

Optionally, the catalyst according to a preferred embodiment of the present invention may also include at least one promoter known to those skilled in the art. Suitable promoters vary with the catalytic metal and may be selected from Groups 1-15 of the Periodic Table of the Elements. By way of example and not limitation, when the catalytic metal is cobalt, suitable promoters include Group 1 elements such as potassium(K), lithium (Li), sodium (Na), and cesium (Cs), Group 2 elements such as calcium (Ca), magnesium (Mg), strontium (Sr), and barium (Ba), Group 3 elements such as scandium (Sc), yttrium (Y), and lanthanum (La), Group 4 elements such as (titanium) (Ti), zirconium (Zr), and hafnium (Hf), Group 5 elements such as vanadium (V), niobium (Nb), and tantalum (Ta), Group 6 elements such as molybdenum (Mo) and tungsten (W), Group 7 elements such as rhenium (Re) and manganese (Mn), Group 8 elements such as ruthenium (Ru) and osmium (Os), Group 9 elements such as rhodium (Rd) and iridium (Ir), Group 10 elements such as platinum (Pt) and palladium (Pd), Group 11 elements such as silver (Ag) and copper (Cu), Group 12 elements, such as zinc (Zn), cadmium (Cd), and mercury (Hg), Group 13 elements, such as gallium (Ga), indium (In), thallium (Ti), and boron (B), Group 14 elements such as tin (Sn) and lead (Pb), and Group 15 elements such as phosphorus (P), bismuth (Bi), and antimony (Sb). When the catalytic metal is cobalt, the promoter is preferably selected from among rhenium, ruthenium, platinum, palladium, boron, silver, and combinations thereof.

When the catalyst includes rhenium, the rhenium is preferably present in the catalyst in an amount between about 0.001 and about 5% by weight, more preferably between about 0.01 and about 2% by weight, most preferably between about 0.2 and about 1% by weight.

When the catalyst includes ruthenium, the ruthenium is preferably present in the catalyst in an amount between about 0.0001 and about 5% by weight, more preferably between about 0.001 and about 1% by weight, most preferably between about 0.01 and about 1% by weight.

When the catalyst includes platinum, the platinum is preferably present in the catalyst in an amount between about 0.00001 and about 5% by weight, more preferably between about 0.0001 and about 1% by weight, and most preferably between about 0.0005 and 1% by weight. It will be understood that each of the ranges, such as of ratio or weight %, herein is inclusive of its lower and upper values.

When the catalyst includes palladium, the palladium is preferably present in the catalyst in an amount between about 0.001 and about 5% by weight, more preferably between about 0.01 and about 2% by weight, most preferably between about 0.2 and about 1% by weight.

When the catalyst includes silver, the catalyst preferably has a nominal composition including from about 0.05 to about 10 wt % silver, more preferably from about 0.07 to about 7 wt % silver, still more preferably from about 0.1 to about 5 wt % silver.

When the catalyst includes boron, the catalyst preferably has a nominal composition including from about 0.025 to about 2 wt % boron, more preferably from about 0.05 to about 1.8 wt. % boron, still more preferably from about 0.075 to about 1.5 wt % boron.

The most preferred method of preparation may vary among those skilled in the art, depending for example on the desired catalyst particle size. Those skilled in the art are able to select the most suitable method for a given set of requirements. By way of illustration and not limitation, such methods include impregnating the catalytically active compounds or precursors onto a support, extruding one or more catalytically active compounds or precursors together with support material to prepare catalyst extrudates, and/or precipitating the catalytically active compounds or precursors onto a support. Accordingly, the supported catalysts of the present invention may be used in the form of powders, particles, pellets, monoliths, honeycombs, packed beds, foams, and aerogels.

A preferred method of preparing a supported metal catalyst (e.g., a supported cobalt catalyst) is by incipient wetness impregnation of the support with a solution of a soluble metal salt such as nitrate, acetate, acetylacetonate or the like. The precursor salt is dissolved in a suitable solvent such as water, methanol or ethanol and impregnated on the support. The impregnated support is dried and reduced with a hydrogen containing gas. In another preferred method, the impregnated support is dried, oxidized with air or oxygen and reduced with a hydrogen containing gas. The oxidation preferably occurs at elevated temperature, such that the oxidation includes calcination.

Thus, present methods of making Fischer-Tropsch catalysts include, for example, impregnation of a support with a solution containing at least one precursor of a catalytic metal and optionally at least one precursor of a promoter, followed by drying the impregnated support, preferably followed by calcination in flowing air. The loading of catalytic metal and any optional promoter on a support may proceed by multistep impregnation, such as by two or three impregnation steps. Each impregnation step may include impregnation of any one or combination of catalytic metal and promoter. Preferably, impregnation proceeds by the known method of incipient wetness, in a small, minimal amount of solvent is used. The solvent may be water, or may be an organic solvent, such as acetone, according to the solubility of a precursor. Further, each precursor may be dissolved in a different solvent, before combining the solutions for impregnation. Each step of impregnating the support to form a catalyst is preferably followed by drying the catalyst, preferably followed by calcining the catalyst in air.

Alternatively, another method involves preparing the supported metal catalyst from a molten metal salt, such as a molten metal nitrate. Still alternatively, the support can be impregnated with a solution of a zero valence metal precursor, in a suitable organic solvent (e.g. toluene). A hydrogen reduction step may not be necessary if the catalyst is prepared with zero valent metal.

Typically, at least a portion of the metal(s) of the catalytic metal component of the catalysts of the present invention is present in a reduced state (i.e., in the metallic state). Therefore, it is normally advantageous to activate the catalyst prior to use by a reduction treatment, in the presence of hydrogen at an elevated temperature. Typically, the catalyst is treated with a hydrogen containing gas at a temperature in the range of from about 75° C. to about 500° C., for about 0.5 to about 24 hours at a pressure of about 1 to about 75 atm. Pure hydrogen may be used in the reduction treatment, as may a mixture of hydrogen and an inert gas such as nitrogen, or a mixtureof hydrogen and other gases as are known in the art, such as carbon monoxide and carbon dioxide. The amount of hydrogen may range from about 1 percent to about 100 percent by volume. Reduction with pure hydrogen and reduction with a mixture of hydrogen and carbon monoxide are preferred methods for reduction.

The feed gases charged to the process of the invention comprise hydrogen, or a hydrogen source, and carbon monoxide. H₂/CO mixtures suitable as a feedstock for conversion to hydrocarbons according to the process of this invention can be obtained from light hydrocarbons such as methane by means of steam reforming, partial oxidation, or other processes known in the art. Preferably the hydrogen is provided by free hydrogen, although some Fischer-Tropsch catalysts have sufficient water gas shift activity to convert some water to hydrogen for use in the Fischer-Tropsch process. It is preferred that the molar ratio of hydrogen to carbon monoxide in the feed be greater than 0.5:1 (e.g., from about 0.67 to 2.5). Preferably, when cobalt, nickel, and/or ruthenium catalysts are used, the feed gas stream contains hydrogen and carbon monoxide in a molar ratio of about 2:1. Preferably, when iron catalysts are used the feed gas stream contains hydrogen and carbon monoxide in a molar ratio between about 0.5:1 and 0.67:1 (e.g. about 0.67:1). The feed gas may also contain carbon dioxide. The feed gas stream should contain a low concentration of compounds or elements that have a deleterious effect on the catalyst, such as poisons. For example, the feed gas may need to be pretreated to ensure that it contains low concentrations of sulfur or nitrogen compounds such as hydrogen sulfide, ammonia and carbonyl sulfides.

The feed gas is contacted with the catalyst in a reaction zone. Mechanical arrangements of conventional design may be employed as the reaction zone including, for example, fixed bed, fluidized bed, slurry phase, slurry bubble column or ebullating bed reactors, among others. Accordingly, the preferred size and physical form of the catalyst particles may vary depending on the reactor in which they are to be used.

The Fischer-Tropsch process is typically run in a continuous mode. In this mode, the gas hourly space velocity through the reaction zone typically may range from about 100 volume·reactants/hour/volume·catalyst (v/hr/v) to about 10,000 v/hr/v, preferably from about 300 v/hr/v to about 2,000 v/hr/v. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psig (653 kPa) to about 1000 psig (6994 kPa), preferably from 80 psig (653 kPa) to about 600 psig (4237 kPa), and still more preferably, from about 140 psig (1066 kPa) to about 400 psig (2858 kPa).

The products resulting from the process will have a great range of molecular weights. Typically, the carbon number range of the product hydrocarbons will start at methane and continue to the limit observable by modern analysis, about 50 to 100 carbons per molecule. The process is particularly useful for making hydrocarbons having five or more carbon atoms especially when the above-referenced preferred space velocity, temperature and pressure ranges are employed.

The wide range of hydrocarbons produced in the reaction zone will typically afford liquid phase products at the reaction zone operating conditions. Therefore the effluent stream of the reaction zone will often be a mixed phase stream including liquid and vapor phase products. The effluent stream of the reaction zone may be cooled to effect the condensation of additional amounts of hydrocarbons and passed into a vapor-liquid separation zone separating the liquid and vapor phase products. The vapor phase material may be passed into a second stage of cooling for recovery of additional hydrocarbons. The liquid phase material from the initial vapor-liquid separation zone together with any liquid from a subsequent separation zone may be fed into a fractionation column. Typically, a stripping column is employed first to remove light hydrocarbons such as propane and butane. The remaining hydrocarbons may be passed into a fractionation column where they are separated by boiling point range into products such as naphtha, kerosene and fuel oils. Hydrocarbons recovered from the reaction zone and having a boiling point above that of the desired products may be passed into conventional processing equipment such as a hydroprocessing zone (.e.g a hydrocracking zone) in order to reduce their molecular weight down to desired products such as middle distillates and gasoline. The gas phase recovered from the reactor zone effluent stream after hydrocarbon recovery may be partially recycled if it contains a sufficient quantity of hydrogen and/or carbon monoxide.

Without further elaboration, it is believed that one skilled in the art can, using the description herein, utilize the present invention to its fullest extent. The following embodiments are to be construed as illustrative, and not as constraining the scope of the present invention in any way whatsoever.

### EXAMPLES

General Procedure for Batch Testing

Each of the catalyst samples 1-12 and 16 was treated with hydrogen according to the following procedure prior to use in the Fischer-Tropsch reaction. The catalyst sample was placed in a small quartz crucible in a chamber and purged with 500 sccm (8.3×10⁻⁶ m³/s) nitrogen at room temperature for 15 minutes. The sample was then heated under 100 sccm (1.7×10⁻⁶ m³/s) hydrogen at 1° C./minute to 100° C. and held at 100° C. for one hour. The catalysts were then heated at 1° C./minute to 400° C. and held at 400° C. for four hours under 100 sccm (1.7×10⁻⁶ m³/s) hydrogen. The samples were cooled in hydrogen and purged with nitrogen before use.

A 2 mL pressure vessel was heated at 225° C. under 1000 psig (6994 kPa) of H₂:CO (2:1) and maintained at that temperature and pressure for 1 hour. In a typical run, roughly 50 mg of the reduced catalyst and 1 mL of n-octane was added to the vessel. After one hour, the reactor vessel was cooled in ice, vented, and an internal standard of di-n-butylether was added. The reaction product was analyzed on an HP6890 gas chromatograph. Hydrocarbons in the range of C₁₁-C₄₀ were analyzed relative to the internal standard. The lower hydrocarbons were not analyzed since they are masked by the solvent and are also vented as the pressure is reduced.

Catalyst Preparation

Group 3 Metal Oxide Supported Catalysts

### Example 1

Lanthanum oxide (1 g) was dried in air at 200° C. and mixed with cobalt carbonyl (0.6 g) in a glove box. The mixture was then placed in a clean quartz boat in a sealed tube furnace tube and removed from the glove box. Flow through dry nitrogen was purged through the furnace tube and out through a bubbler. The furnace tube was then ramped to 100° C. and held there for 15 minutes and then ramped to 200° C. and held there for 30 minutes. The furnace tube was then cooled and taken to the glove box.

### Example 2

Ceria (1 g) was dried in air at 200° C. and mixed with cobalt carbonyl (0.6 g) in a glove box. The mixture was then placed in a clean quartz boat in a sealed tube furnace tube and removed from the glove box. Flow through dry nitrogen was purged through the furnace tube and out through a bubbler. The furnace tube was then ramped to 100° C. and held there for 15 minutes and then ramped to 200° C. and held there for 30 minutes. The furnace tube was then cooled and taken to the glove box.

Group 6 Metal Oxide Supported Catalysts

### Example 3

Chromia (1 g) was dried in air at 200° C. and mixed with cobalt carbonyl (0.6 g) in a glove box. The mixture was then placed in a clean quartz boat in a sealed tube furnace tube and removed from the glove box. Flow through dry nitrogen was purged through the furnace tube and out through a bubbler. The furnace tube was then ramped to 100° C. and held there for 15 minutes and then ramped to 200° C. and held there for 30 minutes. The furnace tube was then cooled and taken to the glove box.

### Example 4

Chromia (1 g) was dried in flowing nitrogen at 200° C. for 30 minutes. The sample was then sealed, placed in a glove box, and mixed with dicobalt octacarbonyl (0.6 g) and rhenium carbonyl (0.02 g). This mixture was then placed in a clean quartz boat in a tube furnace tube, sealed, and removed from the glove box. Flow through dry nitrogen was purged through the furnace tube and out through a bubbler. The furnace tube was then ramped to 100° C. and held there for 15 minutes and then ramped to 200° C. and held there for 30 minutes. The furnace tube was then cooled and taken to the glove box.

### Example 5

Chromia (1 g) was dried in flowing nitrogen at 200° C. for 30 minutes. The sample was then sealed, placed in a glove box, and mixed with dicobalt octacarbonyl (0.6 g) and ruthenium carbonyl (0.0021 g). This mixture was then placed in a clean quartz boat in a tube furnace tube, sealed, and removed from the glove box. Flow through dry nitrogen was purged through the furnace tube and out through a bubbler. The furnace tube was then ramped to 100° C. and held there for 15 minutes and then ramped to 200° C. and held there for 30 minutes. The furnace tube was then cooled and taken to the glove box.

Group 8 Metal Oxide Supported Catalysts

### Example 6

Ferric oxide (1 g) was dried in air at 200° C. and mixed with cobalt carbonyl (0.6 g) in a glove box. The mixture was then placed in a clean quartz boat in a sealed tube furnace tube and removed from the glove box. Flow through dry nitrogen was purged through the furnace tube and out through a bubbler. The furnace tube was then ramped to 100° C. and held there for 15 minutes and then ramped to 200° C. and held there for 30 minutes. The furnace tube was then cooled and taken to the glove box.

Group 12 Metal Oxide Supported Catalysts

### Example 7

Zinc oxide (1 g) was dried in air at 200° C. and mixed with cobalt carbonyl (0.6 g) in a glove box. The mixture was then placed in a clean quartz boat in a sealed tube furnace tube and removed from the glove box. How through dry nitrogen was purged through the furnace tube and out through a bubbler. The furnace tube was then ramped to 100° C. and held there for 15 minutes and then ramped to 200° C. and held there for 30 minutes. The furnace tube was then cooled and taken to the glove box.

### Example 8

Zinc oxide (1 g) was dried in flowing nitrogen at 200° C. for 30 minutes. The sample was then sealed, placed in a glove box, and mixed with dicobalt octacarbonyl (0.6 g) and rhenium carbonyl (0.02 g). This mixture was then placed in a clean quartz boat in a tube furnace tube, sealed, and removed from the glove box. Flow through dry nitrogen was purged through the furnace tube and out through a bubbler. The furnace tube was then ramped to 100° C. and held there for 15 minutes and then ramped to 200° C. and held there for 30 minutes. The furnace tube was then cooled and taken to the glove box.

### Example 9

Zinc oxide (1 g) was dried in flowing nitrogen at 200° C. for 30 minutes. The sample was then sealed, placed in a glove box, and mixed with dicobalt octacarbonyl (0.6 g) and ruthenium carbonyl (0.0021 g). This mixture was then placed in a clean quartz boat in a tube furnace tube, sealed, and removed from the glove box. Flow through dry nitrogen was purged through the furnace tube and out through a bubbler. The furnace tube was then ramped to 100® C. and held there for 15 minutes and then ramped to 200° C. and held there for 30 minutes. The furnace tube was then cooled and taken to the glove box.

### Example 10

Zinc oxide (1 g) was slurried into molten Co(NO₃)₂.6H₂O (0.9877 g). The slurry was dried at 80° C. The solids were removed from the oven and exposed to air to absorb moisture. The solids were then dried again at 80° C. followed by heating the solids at 0.5° C. per minute to 350° C. and maintaining the solids at this temperature for 18 minutes. The solids were then heated at 0.5° C. per minute to 450° C., and reduced in hydrogen flow at 450° C. for 6 hours. The material was cooled and flushed with nitrogen overnight and then sealed for transport into an inert atmosphere glove box. The recovered catalyst was bottled and sealed for storage inside the glove box until Fischer-Tropsch testing could be completed.

### Example 11

Rhenium heptoxide (0.0130 gm) was dissolved in a small amount of water, added to molten Co(NO₃)₂.6H₂O (0.9877 g) and mixed well to form a solution. Zinc oxide (0.7900) was added to the solution to form a slurry. The slurry was dried at 80° C. The solids were removed from the oven and exposed to air to absorb moisture. The solids were then dried again at 80° C. followed by heating the solids at 0.5° C. per minute to 350° C. and maintaining the solids at this temperature for 18 minutes. The solids were then heated at 0.5° C. per minute to 450° C., and reduced in hydrogen flow at 450° C. for 6 hours. The material was cooled and flushed with nitrogen overnight and then sealed for transport into an inert atmosphere glove box. The recovered catalyst was bottled and sealed for storage inside the glove box until Fischer-Tropsch testing could be completed.

Group 15 Metal Oxide Supported Catalysts

### Example 12

Bismuth oxide (1 g) was dried in air at 200° C. and mixed with cobalt carbonyl (0.6 g) in a glove box. The mixture was then placed in a clean quartz boat in a sealed tube furnace tube and removed from the glove box. Flow through dry nitrogen was purged through the furnace tube and out through a bubbler. The furnace tube was then ramped to 100° C. and held there for 15 minutes and then ramped to 200° C. and held there for 30 minutes. The furnace tube was then cooled and taken to the glove box.

Results of Batch Testing

A C₁₁⁺ productivity (g C₁₁⁺/hour/kg catalyst) was calculated based on the integrated production of the C₁₁-C₄₀ hydrocarbons per kg of catalyst per hour. The logarithm of the weight fraction for each carbon number ln(Wₙ/n) was plotted as the ordinate vs. number of carbon atoms in (Wₙ/n) as the abscissa. From the slope, a value of α was obtained. The results of runs over a variety of catalysts at 225° C. are shown in Table 1. The values for C₁₁⁺ productivity and α reported for Examples 1-2, 4, 5, 6, 8, and 10-12 each represent a single measurement. The values for C₁₁⁺ activity and α results reported for Examples 3 and 9 each represent an arithmetic average of two measurements. The values for C₁₁⁺ productivity and (x reported for Example 7 an arithmetic average of three measurements.

These results show that, surprisingly, the chemical identity of the metal oxide support influences the productivity of the catalyst in the Fischer-Tropsch reaction for diesel fraction (diesel weight range) hydrocarbons, in particular C₁₁⁺ hydrocarbons. Particularly advantageous performance is observed with regard to zinc oxide and chromium oxide supports. Each of these metal oxide supports has the advantage, exemplary of some embodiments of the present invention, that an un-promoted cobalt-based catalyst including the metal oxide support is as active or more active than one or both of a corresponding ruthenium-promoted catalyst and a corresponding rhenium-promoted catalyst.

TABLE 1

| Example | Catalyst Nominal Composition | C₁₁⁺ Productivity | α |
|---|---|---|---|
| 1 | 16% Co/La₂O₃ | 40 | 0.85 |
| 2 | 16% Co/CeO₂ | 50 | 0.79 |
| 3 | 16% Co/Cr₂O₃ | 240 | 0.86 |
| 4 | 16% Co/1% Re/Cr₂O₃ | 190 | 0.88 |
| 5 | 16% Co/0.1% Ru/Cr₂O₃ | 250 | 0.88 |
| 6 | 16% Co/Fe₂O₃ | 180 | 0.85 |
| 7 | 16% Co/ZnO | 290 | 0.87 |
| 8 | 16% Co/1% Re/ZnO | 260 | 0.88 |
| 9 | 16% Co/0.1% Ru/ZnO | 230 | 0.89 |
| 10 | 20% Co/ZnO | 120 | .85 |
| 11 | 20% Co/1% Re/ZnO | 150 | 0.85 |
| 12 | 16% Co/Bi₂O₃ | 30 | 0.88 |

The complete disclosures of all patents, patent documents, and publications cited herein are hereby incorporated by reference in their entirety. Should the disclosure of any of the patents, patent documents, and publications that are incorporated herein conflict with the present specification to the extent that it might render a term unclear, the present specification shall take precedence.

The foregoing detailed description and examples have been given for clarity of understanding only. No unnecessary limitations are to be understood therefrom. While a preferred embodiment of the present invention has been shown and described, it will be understood that variations can be made to the preferred embodiment without departing from the scope of, and which are equivalent to, the present invention. For example, the structure and composition of the catalyst can be modified and the order of process steps may be varied. Further, while the examples have been described with respect to a batch process, the process for producing hydrocarbons may be carried out in continuous mode. Accordingly, the scope of protection is not limited to the embodiments described herein, but is only limited by the claims that follow, the scope of which shall include all equivalents of the subject matter of the claims.

What is claimed is:

1. A process for producing hydrocarbons, comprising:
contacting a feed stream comprising hydrogen and carbon monoxide with a catalyst in a reaction zone maintained at conversion-promoting conditions effective to produce an effluent stream comprising hydrocarbons;
wherein the catalyst comprises a combination of MₐO_{b} and N_{c}O_{d}; wherein a is between 1 and 6, b is between 1 and 6, c is between 1 and 3, d is between 1 and 4, N comprises a first metal selected from the group consisting of Group 2 metals, Group 3 metals, Group 6 metals, Group 8 metals, Group 12 metals, Group 15 metals, and combinations thereof; and M comprises a second metal selected from the Group consisting of Group 8 metals, Group 9 metals, and Group 10 metals, and combinations thereof.

2. The process according to claim 1 wherein the metal N is present in a support and wherein the metal M is deposited on the support.

3. The process according to claim 2 wherein the catalyst is made by a method comprising impregnating the support with a solution containing a precursor containing the metal M.

4. The process according to claim 1 wherein the metal M comprises an iron-group metal.

5. The process according to claim 4 wherein the metal M comprises cobalt.

6. The process according to claim 1 wherein the metal N comprises chromium.

7. The process according to claim 1 wherein the metal N comprises zinc.

8. The process according to claim 1 wherein a is between 1 and 3, b is between 1 and 4, c is between 1 and 2, and d is between 1 and 3.

9. The process according to claim 1, further including a promoter in combination with the metal M.

10. A process for producing hydrocarbons, comprising contacting a feed stream comprising hydrogen and carbon monoxide with a catalyst in a reaction zone maintained at conversion-promoting conditions effective to produce an effluent stream comprising hydrocarbons, wherein the catalyst comprises a support comprising an oxide of a metal selected from the group consisting of Group 2 metals, Group 3 metals, Group 8 metals, Group 12 metals, Group 15 metals, and combinations thereof.

11. The process according to claim 10 wherein the support comprises zinc oxide.

12. The process according to claim 10 wherein the support comprises chromia.

13. The process according to claim 10 wherein the catalyst comprises a catalytic metal deposited on said support, wherein the catalytic metal is selected from the group consisting of the iron-group metals and combinations thereof.

14. The process according to claim 13 wherein the catalytic metal comprises cobalt.

15. The process according to claim 13 wherein the catalytic metal is un-promoted.

16. The process according to claim 13 wherein the catalyst further comprises a promoter.

17. The process according to claim 16 wherein the promoter is selected from the group consisting of ruthenium, rhenium, platinum, palladium, silver, boron, and combinations thereof.

18. The process according to claim 10 wherein the hydrocarbons comprise diesel fraction hydrocarbons.

19. The process according to claim 10 wherein the hydrocarbons comprise C₁₁₊ hydrocarbons.

20. The process according to claim 10 wherein the catalyst comprises a catalytically effective amount of cobalt; wherein the cobalt is un-promoted; and wherein the activity of the catalyst is at least the activity of a comparative catalyst comprising essentially the same amount of cobalt and a promoter selected from the group consisting of ruthenium, rhenium, and combinations thereof.

21. The process according to claim 20 wherein the support comprises an oxide selected from the group consisting of zinc and chromium, and combinations thereof.

22. A process for producing hydrocarbons, comprising:
contacting a feed stream comprising hydrogen and carbon monoxide with a catalyst in a reaction zone maintained at conversion-promoting conditions effective to produce an effluent stream comprising hydrocarbons;
wherein the catalyst comprises:
a support comprising an oxide of a metal selected from the group consisting of zinc, chromium, and combinations thereof; and
a catalytic metal comprising cobalt deposited on the support;
wherein the hydrocarbons comprise diesel fraction hydrocarbons.

23. The process according to claim 22 wherein the support comprises zinc oxide.

24. The process according to claim 22 wherein the support comprises chromia.

25. The process according to claim 22 wherein the catalyst further comprises a promoter.

26. The process according to claim 22 wherein the cobalt is un-promoted.

27. The process according to claim 26 wherein the activity of the catalyst is at least the activity of a comparative catalyst comprising essentially the same amount of cobalt and a promoter selected from the group consisting of ruthenium, rhenium, and combinations thereof.

28. The process according to claim 27 wherein the activity comprises the C₁₁₊ hydrocarbon productivity.
